(19) United States
(12) Patent Application Publication
(10) Pub. No.: US 2020/0360652 A1
(43) Pub. Date: Nov. 19, 2020

(54) DEVICE AND METHOD FOR RELIEVING SLEEP DISORDERS

(71) Applicant: MAGICAL SNORELESS LTD., Kfar Qara (IL)

(72) Inventor: Mahmood KANANA, Kfar Qara (IL)

(73) Assignee: MAGICAL SNORELESS LTD., Kfar Qara (IL)

(21) Appl. No.: 16/762,153

(22) PCT Filed: Nov. 7, 2018
(86) PCT No.: PCT/IL2018/051197

§ 371 Date: May 7, 2020

Related U.S. Application Data
(60) Provisional application No. 62/582,972, filed on Nov. 8, 2017.

(51) Int. Cl. A61M 21/02; A61B 5/00; A61H 23/02

(52) U.S. Cl. CPC A61M 21/02; A61B 5/4809; A61M 2021/0022; A61H 23/0263; A61B 5/4818

(57) ABSTRACT

A noninvasive device for reliving sleep disorders is disclosed. The device may include: one or more vibrating units; at least one sensor; one or more attachment element configured to attach the one or more vibrating units to a user's face; and a controller configured to: receive a first signal indicating a disturbed sleeping from the at least one sensor; and activate the one or more vibrating units to apply vibrations to the face of the user to stimulate at least one branch of the trigeminal nerve.

### TECHNICAL FILED OF THE INVENTION

The invention generally relates to a device and a method for reliving sleep disorders and more precisely to a device and a method for reliving sleep disorders using vibrations.

### BACKGROUND OF THE INVENTION

A sleep disorder such as, sleep breathing disorder, for example, snoring, upper airway resistance and obstructive sleep apnea, is a known medical problem causing abnormal sleep patterns of a person or animal Some sleep disorders may affect normal physical functioning, as well as mental functioning . One of the most common and annoying sleep disorders is sleep apnea which include various pauses in breathing or periods of shallow breathing during sleep. In the most common form, apnea follows loud snoring but also choking and pauses in breathing. During sleeping, the normal breathing cycle may be disturbed due to the collapse of walls of the soft tissue in the throat.

The muscles in the throat tend to relax during sleeping and for people suffering from apnea may fully or partially collapse and block the airway.

One of the most popular treatments for obstructive sleep apnea is Continuous Positive Airway Pressure (CPAP). A CPAP Machine uses air pressure to keep the airway open during sleep. However, CPAP machines are inconvenient and noisy and may pose a sleep disturbance to the user and immediate surroundings. Other common ways to prevent snoring and apnea include mouthpieces and other intrusive and invasive devices that are directed to maintain an open airway during sleep time. These solutions are also inconvenient, and users tend to stop using them after a while. These devices are usually designed for mild apneas.

Other known solutions prevent the user from entering deep sleep levels or pulling the user out of deep sleep, by different stimulations, in order to prevent full relaxation of the muscles, and thus preventing the collapse of the tongue during sleep, and the resulting airway blockage. However, such solutions disturb the user's sleep and may affect the user' s sleep quality.

### SUMMARY OF THE INVENTION

Some aspects of the invention may relate to a noninvasive device for reliving sleep disorders. The device may include: one or more vibrating units; at least one sensor; one or more attachment element configured to attach the one or more vibrating units to a user' s face; and a controller configured to: receive a first signal indicating a disturbed sleeping from the at least one sensor; and activate the one or more vibrating units to apply vibrations to the face of the user to stimulate at least one branch of the trigeminal nerve.

In some embodiments, the controller may be further configured to: receive a signal indicating an undisturbed sleeping state from the at least one sensor; and deactivate the one or more vibrating units. In some embodiments, the signal indicating a disturbed sleeping comprise at least one of: snoring noises, heavy breathing noises, pauses in breathing and periods of shallow breathing during sleep due to sleep apnea. In some embodiments, the one or more vibrating units is configured to vibrate at a frequency selected to cause stimulation of the trigeminal nerve. In some embodiments, the one or more vibrating units may be configured to vibrate at more than one frequency and the controller may further be configured to: select a vibrating frequency; and activate the one or more vibrating units to apply vibrations to the forehead of the user at the selected frequency.

In some embodiments, the controller may further be configured to: determine a first vibration profile, wherein the first vibration profile comprises one or more vibration frequencies, the intensity of the vibration and the vibration duration for each frequency in the one or more vibration frequencies; and activate the one or more vibrating units to apply the determined first vibration profile following the receiving of the first signal indicating a disturbed sleeping state from the at least one sensor. In some embodiments, determining the first vibration profile may include selecting a vibration profile from a plurality of vibration profiles stored in a database associated with the controller.

In some embodiments, the controller may further be configured to: detect a time duration between the receive first signal indicating a disturbed sleeping and a second signal indicating a disturbed sleeping; and determine the second vibration profile based on the detected time duration. In some embodiments, the controller may further be configured to: store detected time durations and corresponding first vibration profiles in a database associated with the controller; and determine the second vibration profile based on the stored detected time durations and the corresponding first vibration profiles. In some embodiments, the device may further include a communication unit for communicating with an external device. In some embodiments, the controller may further be configured to: receive one or more signals indicating a disturbed sleeping from the at least one sensor recorded when the user was at a quieter environment than the user's normal sleeping environment; analyze the received one or more signals to extract a disturbed sleeping typical pattern for the user; and record the typical pattern in a database. In some embodiments, the controller may further be configured to: compare the recorded typical pattern to the received first signal; and activate the vibration unit based on the comparison.

Some aspects of the invention may be related to a noninvasive method of reliving sleep disorders, the method may include: receiving a first signal indicating a disturbed sleeping from at least one sensor included in a device for reassuring undisturbed sleeping; and activating the one or more vibrating units, included in the device, to apply vibrations to the forehead of the user. In some embodiments, the method may further include receiving a signal indicating an undisturbed sleeping from the at least one sensor; and deactivating the one or more vibrating units.

In some embodiments, the method may further include: selecting a vibrating frequency from a plurality of vibrating frequencies; and activating the one or more vibrating units to apply vibrations to the forehead of the user at the selected frequency. In some embodiments, the method may further include: determining a first vibration profile, wherein the first vibration profile comprises one or more vibration frequencies, the intensity of the vibration and the vibration duration for each frequency in the one or more vibration frequencies; and activating the one or more vibrating units to apply the determined first vibration profile following the receiving of the first signal indicating a disturbed sleeping from the at least one sensor. In some embodiments, determining the first vibration profile comprises selecting a vibration profile from a plurality of vibration profiles stored in a database associated with the device.

In some embodiments, the method may further include detecting a time duration between the receive first signal indicating a disturbed sleeping and a second signal indicating a disturbed sleeping; and determining the second vibration profile based on the detected time duration. In some embodiments, the method may further include storing detected time durations and corresponding first vibration profiles in database associated with the device; and determining the second vibration profile based on the stored detected time durations and the corresponding first vibration profiles.

In some embodiments, the method may further include receiving one or more signals indicating a disturbed sleeping from the at least one sensor recorded when the user was at a quieter environment than the user's normal sleeping environment; analyzing the received one or more signals to extract a disturbed sleeping typical pattern for the user; and recording the typical pattern in a database. In some embodiments, the method may further include: comparing the recorded typical pattern to the received first signal; and activating the vibration unit based on the comparison.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a high-level block diagram of a noninvasive device for relieving sleep disorders according to some embodiments of the invention;

FIGS. 1B and 1C are illustration of the attachment element and vibration unit attached to a user's face according to some embodiments of the invention: and

FIG. 2 is a flowchart of a noninvasive method of relieving sleep disorders according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of embodiments of the invention. However, it will be understood by those skilled in the art that the present invention may be practiced without these specific details. In other instances, well-known methods, procedures, and components have not been described in detail so as not to obscure the present invention.

Some aspects of the invention may be related to relieving sleep disorders or reassuring undisturbed sleeping by causing noninvasive stimulation of sensory nerves in order to cause contraction of the muscles in the soft tissues around the airways. A device according to some embodiments of the invention may mildly alter the sleep stage without awaking the patient and may indirectly stimulate cross motor nerves within the same nerve that innervates the muscles in the soft tissues around the airway. For example, a device according to some embodiments of the invention may stimulate at least one branch of the trigeminal nerve by applying vibrations on the skin of a patient in proximity to the at least one branch of the trigeminal nerve. Upon stimulating the at least one branch of the trigeminal nerve the tongue muscle may contract, and the mouth airway may reopen (i.e., the soft palate and the tongue).

In some embodiments, the device may provide two vibrations intensities provided in two stages. The first stage may include mild-gentle vibration at an intensity that does not affect the depth or stages of sleep. In case the mild-gentle vibrations are not effective, the device may provide stronger vibrations that may slightly alter the sleep stage, for example, may prevent the user from entering deep sleep levels or may pull the user out of deep sleep.

The trigeminal nerve is the nerve responsible for motor innervation of 8 muscles: including the 4 muscles of mastication (the masseter, the temporalis and the medial and lateral pterygoids), And other four muscles including the tensor veli palatini, the mylohyoid, the anterior belly of the digastric and the tensor tympani. Excitation of the sensory component of the trigeminal nerve (by applying a stimulator on the face) may increase the tonus of the oropharyngeal muscles (including the base of tongue) which may improve the patency of the oropharyngeal airway and consequently improves the upper airway obstruction symptoms during sleeping time. Accordingly, such a stimulation can be performed by placing a sensory stimulation device according to some embodiments of the invention on the forehead of the user.

Applying a sensory stimulation device, according to some embodiments of the invention, laterally on the face above the muscles of mastication (e.g., from two sides of mouth/jaw) specifically the masseter muscle may lead to similar effect on the airway patency and the sleep obstruction symptoms. In some embodiments, the effect may occur through the following mechanism: The skin area above the masseter muscle is sensory innervated by the mandibular branch of the trigeminal nerve (V3), thus stimulating this area may cause similar ascending sensory-descending motor reaction as described previously with respect to the direct stimulation of trigeminal nerve. In some embodiments, the muscles spindle receptors within the muscle tissue may also be stimulated by the stimulator vibration and activate neural pathways that lead to increasing the tonus of the muscles in the upper airway pathway, these spindles are also innervated by the trigeminal nerve.

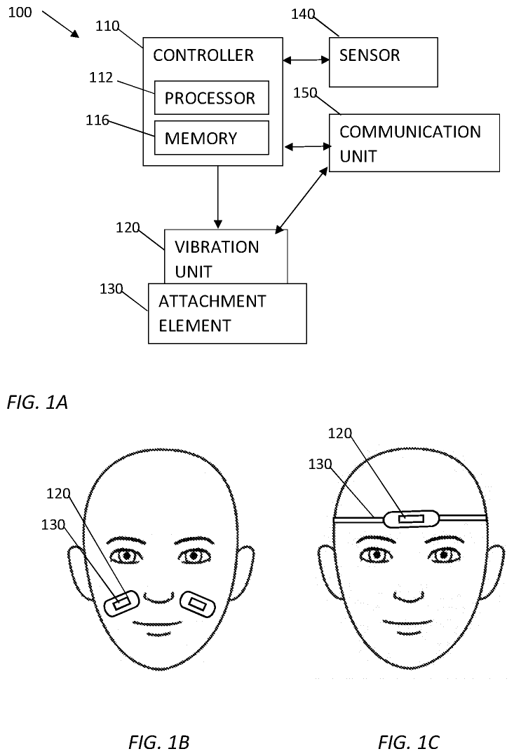

Reference is made to FIG. 1 which is high level block diagram of a noninvasive device for relieving sleep disorders according to some embodiments of the invention. A noninvasive device 100 for relieving sleep disorders may include a controller 110, one or more vibrating units (vibrators) 120, an attachment element 130 configured to attach one or more vibrating units 120 to a user' s forehead or from both sides of the user's mouth/jaw and at least one sensor 140. In some embodiments, device 100 may further include a communication unit 150 for communicating with an external device, such as a smartphone, a tablet, a PC and the like. Communication unit 150 may include any wireless or wired communication unit, for example, 2.4-GHz RF transceiver compatible with Bluetooth low energy (BLE) 4.2 specification.

In some embodiments, controller 110 may include a processor 112 that may be any computing platform such as a chip (e.g., a microcontroller including: 48-MHz clock speed and supports over-the-air upgrade (OTA)) and a memory 114 (e.g., a serial flash memory 16 [mB]). Processor 112 or other processors may be configured to carry out methods according to embodiments of the present invention by for example executing instructions stored in a memory such as memory 114. Memory 114 may be or may include, for example, a memory chip, a Flash memory, a volatile memory, a non-volatile memory, or any other suitable memory units or storage units.

Vibrating unit 120 may be or may include any movable element that may cause vibrations on a skin of a patient when attached to the skin. Vibrating unit 120 may further include a motor (e.g., an electric motor) and gear or a piezoelectric device. For example, vibrating unit 120 may be a coin vibration motor having a body diameter of 10 mm, thickness of 3 mm, operating voltage: 2.7-3.3V, rated vibration speed of 9,000 RPM and vibe force of 0.6 G. Vibrating unit 120 may vibrate at more than one frequency and/or more than one intensity (e.g., vibe force) and controller 110 may be configure to control vibrating unit 120 to vibrate at a specific frequency and/or a specific intensity. In some embodiments, controller 110 may be configured to activate or deactivate one or more vibrating units 120.

Attachment element 130 may be any element that may be configured to attach one or more vibrating units 120 to a user's face (e.g., at the forehead, cheeks, two sides of the mouth/jaw, etc.) as illustrated in FIGS. 1B and 1C. Attachment element 130 may attach one or more vibrating units 120 such that one or more vibrating units 120 may cause vibrations in the user's skin. Attachment element 130 may include, a warble element (e.g., a hat), an elastic strip (as illustrated in FIG. 1C), stickers (as illustrated in FIG. 1B), bandages, and the like.

Sensor 140 may be any sensor that is configured to sense sounds made by the user during sleeping. Sensor 140 may be configured to detect snoring sounds, breathing sounds, choking sounds and the like. Sensor 140 may be, for example, a microphone, such as, a MEMS Microphone having: a sensitivity of −42 dBV and flat frequency response from 100 Hz to 15 kHz. Sensor 140 may be placed in proximity to vibrating units 120 on the user's face, attached to attachment element 130. Alternatively or additionally, at least one sensor 140 may be placed elsewhere in any place surrounding the user that may allow sensor 140 to sense sounds made by the user during sleeping, for example, on a bedside table. In some embodiments at least one sensor 140 may send to controller 110 a signal indicating a disturbed sleeping. In some embodiments, a signal indicating a disturbed sleeping may include at least one of: snoring sounds, heavy breathing sounds, pauses in breathing and periods of shallow breathing during sleeping.

In some embodiments, device 100 may further include a power source (not illustrated) such as a battery, for providing electric power to various components of device 100, such as controller 110, vibration unit 120, sensor 140 and communication unit 150. In some embodiments, at least some of the electronic components of device 100 may be include/connected to a printed circuit board (PCB) attached to attachment element 130. In some embodiments, the battery may be included in the PCB.

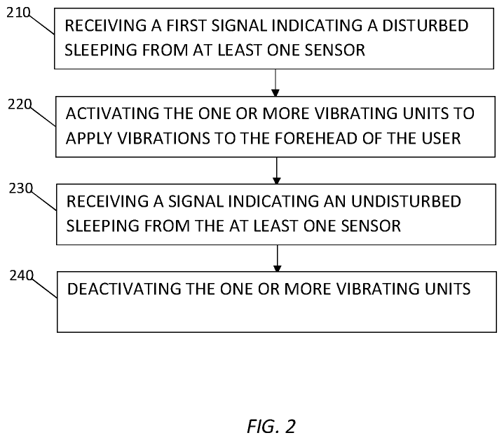

Reference is now made to FIG. 2 which is a flowchart of noninvasive method of relieving sleep disorders according to some embodiments of the invention. The method of FIG. 2 may be performed by a noninvasive device for relieving sleep disorders, such as device 100. In step 210, a first signal indicating disturbed sleeping may be received, for example, by controller 110, from at least one sensor (e.g., sensor 140) included in a device for relieving sleep disorders (e.g., device 100). In some embodiments, the first signal indicating a sleeping disorder may include at least one of: snoring sounds, heavy breathing sounds, pauses in breathing and periods of shallow breathing during sleep.

In step 220, one or more vibrating units (e.g., units 120) may be activated, by controller 110, to apply vibrations to predetermined locations on the user's face, for example, the forehead of the user, or in proximity to the muscles of mastication at two sides of the mouth or the nous, to stimulate at least one sensory nerve of the face, for example, a branch of the trigeminal nerve. In some embodiments, the vibration frequency/ vibration intensity may be selected prior to the activation of the one or more vibrating units 120, for example, by the user or by a professional (e.g., a doctor). Controller 110 may receive the selection from a user interface associated with device 100 or the external device, via communication unit 150.

In some embodiments, controller 110 may activate one or more vibrating units 120 to apply vibrations to the predetermined location on the face of the user at the selected frequency/intensity. In some embodiments, the vibration frequency and/or vibration intensity (e.g., vibe force) may be adapted for the user dynamically. For example, by selecting a low starting frequency and/or low starting intensity and raising the frequency/intensity until an effect is noted (e.g., the signals, such as snoring sounds, indicating a sleep disorder or disturbance, stop). According to some embodiments, the effective frequency rages and/or effective vibe force ranges for a specific user may be recorded (e.g., in memory 114 in FIG. 1) and the starting frequency/vibe force as well as other parameters of the operation scheme (or profile), such as the duration of each vibration cycle, the number of vibration units activated and their location, and the like, may be continuously monitored and adapted to the user based on the detected effective parameters' values. In some embodiments, the controller may control the power or voltage provided to vibration unit 120 in order to change the provided vibration profile (e.g., provided frequency and/or intensity).

In some embodiments, a first vibration profile may be determined or received by controller 110. For example, the first vibration profile may include one or more vibration frequencies, the intensity of the vibration and the vibration duration for each frequency in the one or more vibration frequencies. The first vibration profile may be determined by controller 110, for example, based on signals received from sensor 140 (e.g., heavy breathing sounds, pauses in breathing and the like), based on medical data related to the user, dynamically adapted until an effect is noted and the like. For example, the first vibration profile may include providing 2.7 volts to a coin vibration motor for 300 milliseconds. The provided voltage may determine the provided intensity.

Additionally or alternatively, the first vibration profile may be received from the external device, for example, a smartphone of the user, via communication unit 150. In some embodiments, controller 110 may activate one or more vibrating units 120 to apply the first vibration profile following the receiving of the first signal indicating a disturbed sleeping state from at least one sensor 140. In some embodiments, the first vibration profile may be selected from a plurality of vibration profiles stored in a database associated with the controller, for example, in memory 114 or in a database associated with the external device.

In some embodiments, controller 110 may detect a time duration between the received first signal indicating a disturbed sleeping and a second signal indicating a disturbed sleeping received from at least one sensor 140. In some embodiments, controller 110 may determine a second vibration profile based on the detected time duration. For example, if the time duration is relatively short, e.g., 1-2 minutes, controller 110 may increase at least one of: the vibration frequencies, the intensity of the vibration and the vibration duration in order to cause longer durations of undisturbed sleeping. For example, controller 110 may increase the intensity be and duration by providing 3 volts to a coin vibration motor for 400 milliseconds.

In step 230, a signal indicating an undisturbed sleeping may be received from the at least one sensor. For example, sensor 140 may detect the sound of an easy relaxed breathing. In step 240, one or more vibrating units 120 may be deactivated.

In some embodiments, the method may further include storing detected time durations and corresponding first vibration profiles in database associated with the device, for example, memory 114 or an external memory in communication with controller 110. The process may include learning the behavior and response of the user to applications of the vibration profiles in a period of time (e.g., two weeks). The controller may then determine a second vibration profile, if the first vibration profile did not achieve the required undisturbed sleeping. For example, controller 110 may select a different vibration frequency, increase/decrease the intensity of the vibration and/or change the vibration duration.

In some embodiments, the method may further include recording signals indicating a disturbed sleeping over a period of time and analyzing the recorded signal to identify repeated/typical patterns in the recorded signals. In some embodiments, controller 110 may receive and store the vibration profiles at which the undisturbed sleeping has being achieved for each signal indicating disturbed sleeping (e.g., snoring/heavy breading sounds). Controller 110 or any other controller may use the stored data in order to determine the required vibration profile for the received signal indicating disturbed sleeping, by comparing received signal with previously stored signals or group of signals and the corresponding stored vibration profiles.

In some embodiments, an initial recording of one or more signals indicating a disturbed sleeping may take place in substantially quiet environment, for example, in a sleep laboratory which is quieter than the user's normal sleeping environment. Controller 110 may then analyze the received one or more signals to extract a disturbed sleeping typical pattern for the user. Controller 110 may record the typical pattern in a database, such as, memory 116 or any other database associated with controller 110. In some embodiments, the recorded typical pattern may be compared to the received first signal. The comparison may allow to filter from the real time first/second signals background noises, therefore, vibration unit 120 may be activated based on the comparison.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A noninvasive device for reliving sleep disorders, comprising:
one or more vibrating units;
at least one sensor;
one or more attachment element configured to attach the one or more vibrating units to a user's face; and
a controller configured to:
receive a first signal indicating a disturbed sleeping from the at least one sensor; and
activate the one or more vibrating units to apply vibrations to the face of the user to stimulate at least one branch of the trigeminal nerve.

2. The device of claim 1, wherein the controller is further configured to:
receive a signal indicating an undisturbed sleeping state from the at least one sensor; and
deactivate the one or more vibrating units.

3. The device of claim 1, wherein the signal indicating a disturbed sleeping comprise at least one of: snoring noises, heavy breathing noises, pauses in breathing and periods of shallow breathing during sleep due to sleep apnea.

4. The device of claim 1, wherein the one or more vibrating units is configured to vibrate at a frequency selected to cause stimulation of the trigeminal nerve.

5. The device of claim 1, wherein the one or more vibrating units is configured to vibrate at more than one frequency and the controller is further configured to:
select a vibrating frequency; and
activate the one or more vibrating units to apply vibrations to the forehead of the user at the selected frequency.

6. The device of claim 1, wherein the controller is further configured to:
determine a first vibration profile, wherein the first vibration profile comprises one or more vibration frequencies, the intensity of the vibration and the vibration duration for each frequency in the one or more vibration frequencies; and
activate the one or more vibrating units to apply the determined first vibration profile following the receiving of the first signal indicating a disturbed sleeping state from the at least one sensor.

7. The device of claim 6, wherein determining the first vibration profile comprises selecting a vibration profile from a plurality of vibration profiles stored in a database associated with the controller.

8. The device of claim 6, wherein the controller is further configured to:
detect a time duration between the receive first signal indicating a disturbed sleeping and a second signal indicating a disturbed sleeping; and
determine the second vibration profile based on the detected time duration.

9. The device of claim 8, wherein the controller is further configured to:
store detected time durations and corresponding first vibration profiles in a database associated with the controller; and
determine the second vibration profile based on the stored detected time durations and the corresponding first vibration profiles.

10. (canceled)

11. The device of claim 1, wherein the controller is further configured to:
receive one or more signals indicating a disturbed sleeping from the at least one sensor recorded when the user was at a quieter environment than the user's normal sleeping environment;
analyze the received one or more signals to extract a disturbed sleeping typical pattern for the user; and
record the typical pattern in a database.

12. The device of claim 11, wherein the controller is further configured to:
compare the recorded typical pattern to the received first signal; and
activate the vibration unit based on the comparison.

13. A noninvasive method of reliving sleep disorders, comprising:
receiving a first signal indicating a disturbed sleeping from at least one sensor included in a device for reassuring undisturbed sleeping; and
activating the one or more vibrating units, included in the device, to apply vibrations to the forehead of the user.

14. The method of claim 13, further comprising:
receiving a signal indicating an undisturbed sleeping from the at least one sensor; and
deactivating the one or more vibrating units.

15. A method of claim 13, further comprising:
selecting a vibrating frequency from a plurality of vibrating frequencies; and
activating the one or more vibrating units to apply vibrations to the forehead of the user at the selected frequency.

16. A method of claim 13, further comprising:
determining a first vibration profile, wherein the first vibration profile comprises one or more vibration frequencies, the intensity of the vibration and the vibration duration for each frequency in the one or more vibration frequencies; and
activating the one or more vibrating units to apply the determined first vibration profile following the receiving of the first signal indicating a disturbed sleeping from the at least one sensor.

17. The method of claim 16, wherein determining the first vibration profile comprises selecting a vibration profile from a plurality of vibration profiles stored in a database associated with the device.

18. The method of claim 18, further comprising:
detecting a time duration between the receive first signal indicating a disturbed sleeping and a second signal indicating a disturbed sleeping; and
determining the second vibration profile based on the detected time duration.

19. The method of claim 18, further comprising:
storing detected time durations and corresponding first vibration profiles in database associated with the device; and
determining the second vibration profile based on the stored detected time durations and the corresponding first vibration profiles.

20. A method of claim 13, further comprising:
receiving one or more signals indicating a disturbed sleeping from the at least one sensor recorded when the user was at a quieter environment than the user's normal sleeping environment;
analyzing the received one or more signals to extract a disturbed sleeping typical pattern for the user; and
recording the typical pattern in a database.

21. The method of claim 20, further comprising:
comparing the recorded typical pattern to the received first signal; and
activating the vibrator based on the comparison.
